(19) United States
(12) Patent Application Publication
(10) Pub. No.: US 2024/0013911 A1
(43) Pub. Date: Jan. 11, 2024

(54) System and Method for Monitoring Health Signals of Patients

(71) Applicant: Clay Tramel, Addison, TX (US)

(72) Inventor: Clay Tramel, Addison, TX (US)

(21) Appl. No.: 18/348,328

(22) Filed: Jul. 6, 2023

Related U.S. Application Data
(60) Provisional application No. 63/367,792, filed on Jul. 6, 2022.

(51) Int. Cl. G16H 40/67; G16H 50/30; G16H 15/00; G16H 20/70

(52) U.S. Cl. CPC G16H 40/67; G16H 50/30; G16H 15/00; G16H 20/70; G06Q 10/1095

(57) ABSTRACT

A monitoring system for facilitating treatment of a patient user group in an addiction treatment program. The monitoring system comprises at least a wearable mobile computer, a server, a device application and a server application. The device application is on the wearable mobile computer. The server application is on the server. The server application comprises at least a caregiver application for reporting a status of the patient user group to a caregiver user group of the monitoring system. The caregiver user group can comprise therapists and accountability partners. The monitoring system collects a wearable health data for each among the patient user group which are wearing the wearable mobile computer. The monitoring system can record the wearable health data using sensors on the wearable mobile computer. The monitoring system is adapted at tracking changes in the wearable health data to predict a relapse or pre-relapse behavior.

### CROSS-REFERENCE TO RELATED APPLICATIONS

This application claims benefit to and incorporates by reference US provisional patent application 63/367,792 filed 2022 Jul. 6.

### STATEMENT REGARDING FEDERALLY SPONSORED RESEARCH OR DEVELOPMENT (IF APPLICABLE)

Not applicable.

### REFERENCE TO SEQUENCE LISTING, A TABLE, OR A COMPUTER PROGRAM LISTING COMPACT DISC APPENDIX (IF APPLICABLE)

Not applicable.

### BACKGROUND OF THE INVENTION

No prior art is known to the Applicant.

### BRIEF SUMMARY OF THE INVENTION

A monitoring system for assessing a condition of and facilitating treatment of a patient user group in an addiction treatment program. Said monitoring system comprises at least a wearable mobile computer, a server, a device application and a server application. Said device application is on said wearable mobile computer. Said server application is on said server. Said server application comprises at least a caregiver application for reporting a status of said patient user group to a caregiver user group of said monitoring system. Said caregiver user group can comprise therapists and accountability partners. Said monitoring system collects a wearable health data for each among said patient user group which are wearing said wearable mobile computer. Said monitoring system can record said wearable health data using sensors on said wearable mobile computer. Said monitoring system is adapted at tracking changes in said wearable health data to predict a relapse or pre-relapse behavior. Said monitoring system is configured to report a portion of said wearable health data to said caregiver application and thereafter to a portion of said caregiver user group. Said wearable health data comprises a portion of a location monitor data, a heart rate monitor data, a light monitor data, a step monitor data, a device worn data, a sleep data, and a respiration monitor. Said respiration monitor is attached to said patient user group to track respiration rates and similar data among said wearable health data.

Said monitoring system for assessing a condition of and facilitating treatment of said patient user group in an addiction treatment program. Said monitoring system comprises at least said wearable mobile computer, said server, said device application and said server application. Said device application is on said wearable mobile computer. Said server application is on said server. Said server application comprises at least said caregiver application for reporting a status of said patient user group to said caregiver user group of said monitoring system. Said caregiver user group can comprise therapists and accountability partners. Said monitoring system collects said wearable health data for each among said patient user group which are wearing said wearable mobile computer. Said monitoring system can record said wearable health data using sensors on said wearable mobile computer. Said monitoring system is adapted at tracking changes in said wearable health data to predict a relapse or pre-relapse behavior. Said monitoring system is configured to report a portion of said wearable health data to said caregiver application and thereafter to a portion of said caregiver user group.

Said monitoring system for assessing a condition of and facilitating treatment of said patient user group in an addiction treatment program. Said monitoring system comprises at least said wearable mobile computer, said server, said device application and said server application. Said device application is on said wearable mobile computer. Said server application is on said server. Said server application comprises at least said caregiver application for reporting a status of said patient user group to said caregiver user group of said monitoring system. Said caregiver user group can comprise therapists and accountability partners. Said monitoring system collects said wearable health data for each among said patient user group which are wearing said wearable mobile computer. Said monitoring system can record said wearable health data using sensors on said wearable mobile computer. Said monitoring system is adapted at tracking changes in said wearable health data to predict a relapse or pre-relapse behavior. Said monitoring system is configured to report a portion of said wearable health data to said caregiver application and thereafter to a portion of said caregiver user group. Said wearable health data comprises a user location data, a blacklist locations and a whitelist locations. Said monitoring system is configured for collecting said user location data. Comparing said user location data to said blacklist locations and said whitelist locations. Determining if the users' location is within said user location data or said blacklist locations. Reporting on the status of a user.

### BRIEF DESCRIPTION OF THE SEVERAL VIEWS OF THE DRAWING

FIG. 1 illustrates a flow chart view of a network diagram 102.

FIGS. 2A, 2B, 2C, 2D and 2E illustrate a perspective overview view of a mobile phone 200a, a personal computer 200b, a tablet 200c, a wearable mobile computer 106c and a smartphone 222.

FIG. 3 illustrates view of an address space 302.

FIG. 4 illustrates embodiments for collecting and data with a communication hardware 306e 308 and a data storage 110.

FIG. 5 illustrates a flow chart view of said data storage 110, a device application 502, and a server application 506.

FIG. 6 illustrates a flow chart view of said data storage 110, said device application 502, and said server application 506.

FIG. 7 illustrates a flow chart view of a monitoring system 100.

FIG. 8 illustrates a flow chart view of a monitoring data illustration 800.

FIG. 9 illustrates a flow chart view of monitoring procedure 900.

FIG. 10 illustrates a flow chart view of location monitoring procedure 1000.

FIG. 11 illustrates a view of a location monitoring flowchart 1100.

FIG. 12 illustrates a view of an encouragement method 1200.

FIG. 13 illustrates a view of an TO Event monitoring method 1300.

FIG. 14 illustrates a view of a notification method 1400.

FIGS. 15A and 15B illustrate a view of an index geographical chart 1500 and a dynamic index update procedure 1518.

FIG. 16 illustrates a view of a motion detection method 1600.

FIG. 17 illustrates a view of a method of creating a recovery score card 1700.

FIG. 18 illustrates a view of a recovery scorecard 1802.

FIG. 19 illustrates a view of an overview chart 1900.

FIG. 20 illustrates an integrity ally module 2000 of said monitoring system 100.

FIG. 21 illustrates a detox ally module 2100 as a flow chart.

FIG. 22 illustrates a protective ally module 2200 of said monitoring system 100.

### DETAILED DESCRIPTION OF THE INVENTION

The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

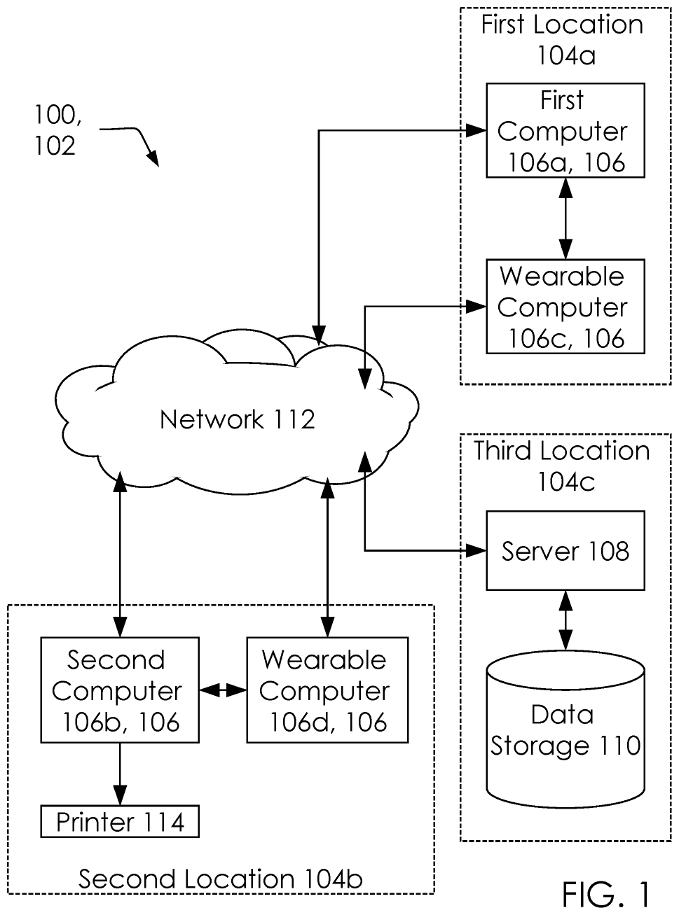

FIG. 1 illustrates a flow chart view of a network diagram 102.

In one embodiment, said network diagram 102 can comprise one or more locations 104, one or more mobile computers 106, a server 108, a data storage 110, a network 112 and a printer 114.

In one embodiment, said one or more mobile computers 106 can comprise a first mobile computer 106a, a second mobile computer 106b, a wearable mobile computer 106c and said wearable mobile computer 106c.

In one embodiment, said one or more locations 104 can comprise a first location 104a, a second location 104b, a third location 104c and a monitoring center 104d.

In one embodiment, said data storage 110 can comprise a data storage 110a.

In one embodiment, a monitoring system 100 can comprise said one or more mobile computers 106, said server 108, said data storage 110, said network 112, said printer 114 and one or more wearable computers 116.

In one embodiment, said one or more wearable computers 116 can comprise a first wearable computer 116a and a second wearable computer 116b.

In one embodiment, said monitoring system 100 can comprise a system for the care of people through monitoring, encouraging and reporting dangerous behavior, as outlined herein. Illustrated herein as said network diagram 102 can comprise a diagram of some of the parts and persons who may use said monitoring system 100.

In one embodiment, said printer 114 can be hardwired to said first mobile computer 106a (not illustrated here), or said printer 114 can connect to one of said one or more mobile computers 106 (such as said third computer 106c, illustrated) said network 112.

Said network 112 can be a local network (LAN), a wide area network (WAN), a piconet, or a combination of LANs, WANs, or piconets. One illustrative LAN is said network 112 within a single business. One illustrative WAN is the Internet.

In one embodiment, said server 108 represents at least one, but can be many servers, each connected to said network 112. Said server 108 can connect to said data storage 110. Said data storage 110 can connect directly to said server 108, as shown in FIG. 1, or may exist remotely on said network 112. In one embodiment, said data storage 110 can comprise any suitable long-term or persistent storage device and, further, may be separate devices or the same device and may be collocated or distributed (interconnected via any suitable communications network).

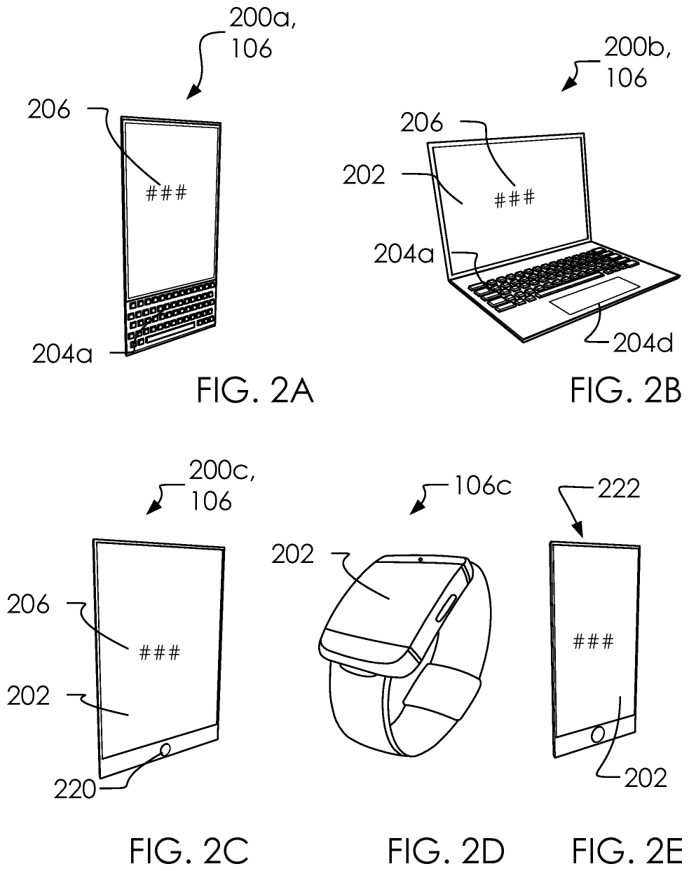

FIGS. 2A, 2B, 2C, 2D and 2E illustrate a perspective overview view of a mobile phone 200a, a personal computer 200b, a tablet 200c, said wearable mobile computer 106c and a smartphone 222.

In one embodiment, said tablet 200c can comprise a screen 202, a data 206 and a home button 220.

In one embodiment, said personal computer 200b can comprise said screen 202, one or more input devices 204, a keyboard 204a, a trackball 204b and said data 206.

In one embodiment, said mobile phone 200a can comprise said keyboard 204a, said trackball 204b and said data 206.

In one embodiment, said one or more input devices 204 can comprise said keyboard 204a, said trackball 204b, one or more cameras 204c and a track pad 204d.

In one embodiment, said one or more mobile computers 106 can comprise said mobile phone 200a, said personal computer 200b, said tablet 200c, a smart watch 200d and a smart phone 200e.

In one embodiment, said monitoring system 100 can comprise said smart watch 200d.

In the last several years, the useful definition of a computer has become more broadly understood to include mobile phones, tablet computers, laptops, desktops, and similar. For example, Microsoft®, have merged devices such as said tablet 200c computer and a laptop computer. In one embodiment, said one or more mobile computers 106 each can include, but is not limited to, a laptop (such as said personal computer 200b), desktop, workstation, server, mainframe, terminal, said tablet 200c (such as said tablet 200c), a phone (such as said mobile phone 200a), and/or similar. Despite different form-factors, said one or more computers can have similar basic hardware, such as said screen 202 and said one or more input devices 204 (such as said keyboard 204a, said trackball 204b, said one or more cameras 204c, a wireless—such as RFID—reader, said track pad 204d, and/or said home button 220). In one embodiment, said screen 202 can comprise a touch screen. In one embodiment, said track pad 204d can function similarly to a computer mouse as is known in the art. In one embodiment, said tablet 200c and/or said personal computer 200b can comprise a Microsoft® Windows® branded device, an Apple® branded device, or similar. In one embodiment, said tablet 200c can be an X86 type processor or an ARM type processor, as is known in the art.

Said monitoring system 100 can comprise said data 206, which can be displayed on said one or more mobile computers 106.

In one embodiment, said one or more mobile computers 106 can be used to input and view said data 206. In one embodiment, said data 206 can be input into said one or more computers by taking pictures with one of said one or more camera 204c, by typing in information with said keyboard 204a, or by using gestures on said screen 202 (where said screen 202 is a touch screen). Many other data entry means for devices like said one or more computers are well known and herein also possible with data 206. Said one or more mobile computers 106 can comprise smartphones, smartwatches, tablets, desktop computers, laptops, server, or others as is known in the art.

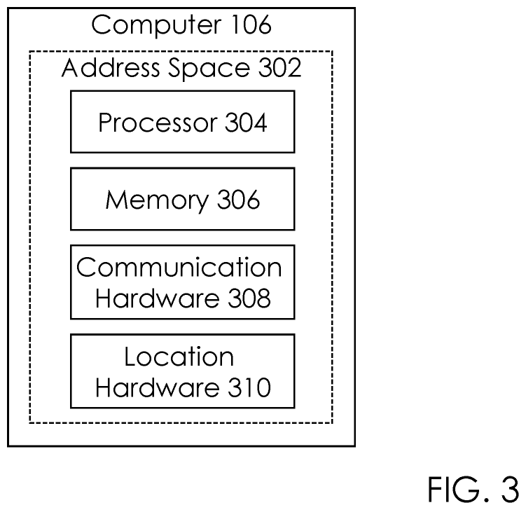

FIG. 3 illustrates view of an address space 302.

In one embodiment, said one or more mobile computers 106 can comprise said address space 302. In one embodiment, said first mobile computer 106a can comprise an address space 302a. In one embodiment, said server 108 can comprise an address space 302e.

In one embodiment, said address space 302 can comprise a processor 304, a memory 306, a communication hardware 308 and a location hardware 310. In one embodiment, said address space 302a can comprise a processor 304a, a memory 306a and a communication hardware 308a. In one embodiment, said address space 302e of said server 108 can comprise a processor 304e, a communication hardware 306e, memory 306e, and a communication hardware 308e.

In one embodiment, said wearable mobile computer 106c can comprise an address space 302d.

Each among said one or more mobile computers 106 and said server 108 can comprise an embodiment of said address space 302. In one embodiment, said address space 302 can comprise said processor 304, said memory 306, and said communication hardware 308e. In one embodiment, said processor 304 can comprise a plurality of processors, said memory 306 can comprise a plurality of memory modules, and said communication hardware 308e can comprise a plurality of communication hardware components.

In one embodiment, said data 206 can be sent to said processor 304; wherein, said processor 304 can perform processes on said data 206 according to a device application 502 stored in said memory 306, as discussed further below. Said processes can include storing said data 206 into said memory 306, verifying said data 206 conforms to a one or more preset standards, or ensuring a required set among said required data 206 has been gathered for said data 206 management system and method. In one embodiment, said data 206 can include data which said one or more computers can populate automatically, such as a date and a time, as well as data entered manually. Once a portion of gathering data has been performed said data 206 can be sent to said communication hardware 306e 308 for communication over said network 112. Said memory 306e 308 can include said network 112 transport processor for packetizing data, communication ports for wired communication, or an antenna for wireless communication. In one embodiment, said data 206 can be collected in one or more computers and delivered to said server 108 through said network 112.

In one embodiment, one among said one or more mobile computers 106 can comprise said address space 302a. Likewise, in one embodiment, said server 108 can comprise said address space 302e.

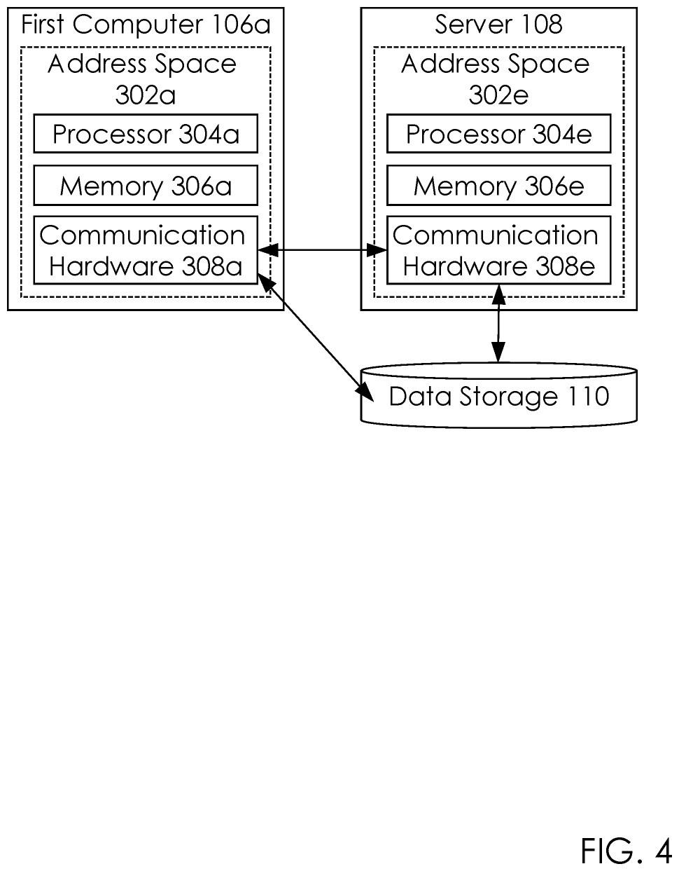

FIG. 4 illustrates embodiments for collecting and data with said communication hardware 306e 308 and said data storage 110.

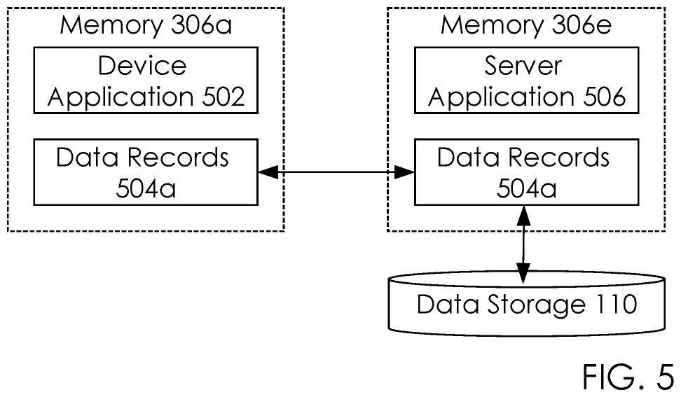

FIG. 5 illustrates a flow chart view of said data storage 110, said device application 502, and a server application 506.

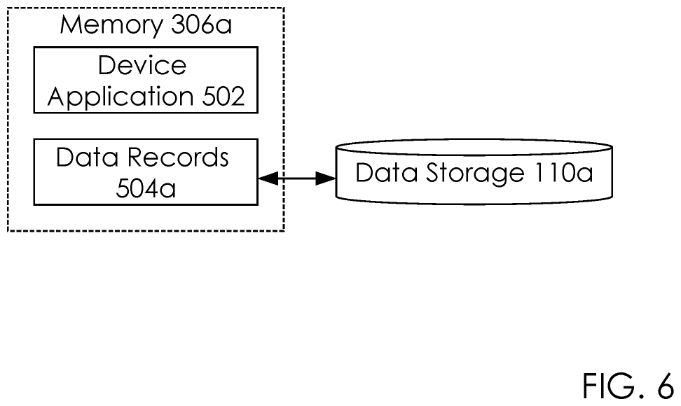

FIG. 6 illustrates a flow chart view of said data storage 110, said device application 502, and said server application 506.

Said monitoring system 100 can comprise said device application 502 and said server application 506, as is known in the art. In one embodiment, said memory 306a can comprise said device application 502. In one embodiment, a memory 306d can comprise said server application 506.

In one embodiment, said monitoring system 100 can comprise a data records 504a.

In one embodiment, said monitoring system 100 can comprise said device application 502 deployed on end user machines and said server 108 application 506 on said server 108.

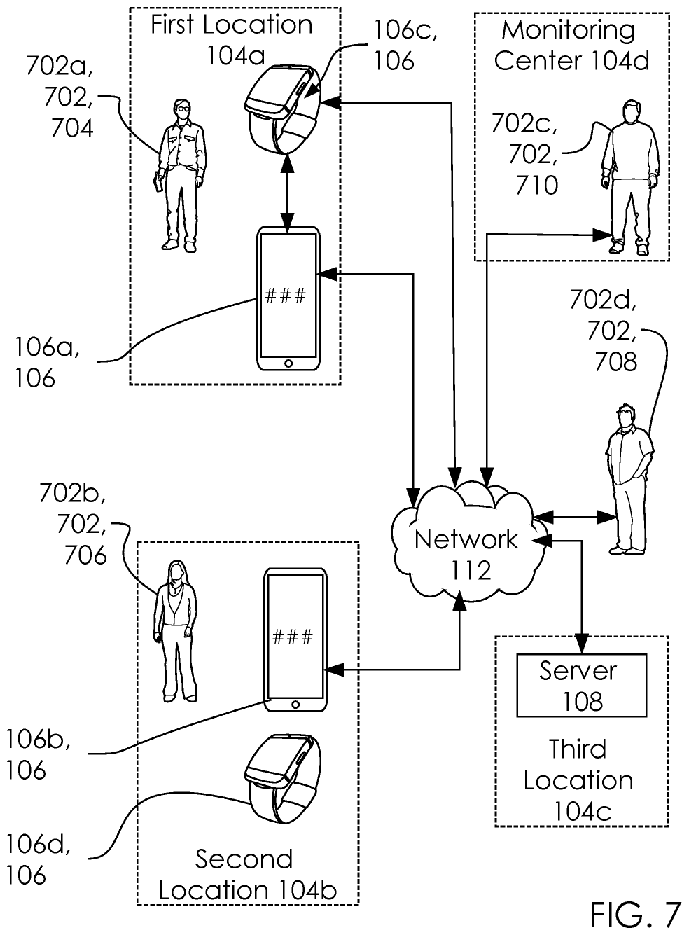

FIG. 7 illustrates a flow chart view of said monitoring system 100.

In one embodiment, one or more users 702 can comprise a first user 702a, a second user 702b, a third user 702c, a fourth user 702d, a patient user group 704, a therapist user group 706, an accountability user group 708 and a caregiver user group 710.

In one embodiment, said monitoring system 100 can be useful in the addiction treatment (“AT”) sector.

In one embodiment, said monitoring system 100 can track said patient user group 704 as they move about and live life.

In one embodiment, said patient user group 704 can wear said wearable mobile computer 106c, or a wearable of different type, as is known in the art. Wherein, said patient user group 704 can wear said wearable mobile computer 106c for the benefits of the current system.

In one embodiment, said monitoring system 100 can report incidents to said monitoring center 104d where immediate action can be taken and said patient user group 704 can be reached. In one embodiment, said monitoring center 104d can comprise said caregiver user group 710 entrusted with data generated by said monitoring system 100.

In one embodiment, said accountability user group 708 can comprise an accountability partner or similar.

In one embodiment, data from said monitoring system 100 can be reviewed by said therapist user group 706 during a clinical visit between said patient user group 704 and said therapist user group 706. In one embodiment, said can receive information on said second mobile computer 106b and/or said wearable mobile computer 106c.

In one embodiment, said monitoring system 100 can send data to said third location 104c to process information on said server 108 before enacting actions.

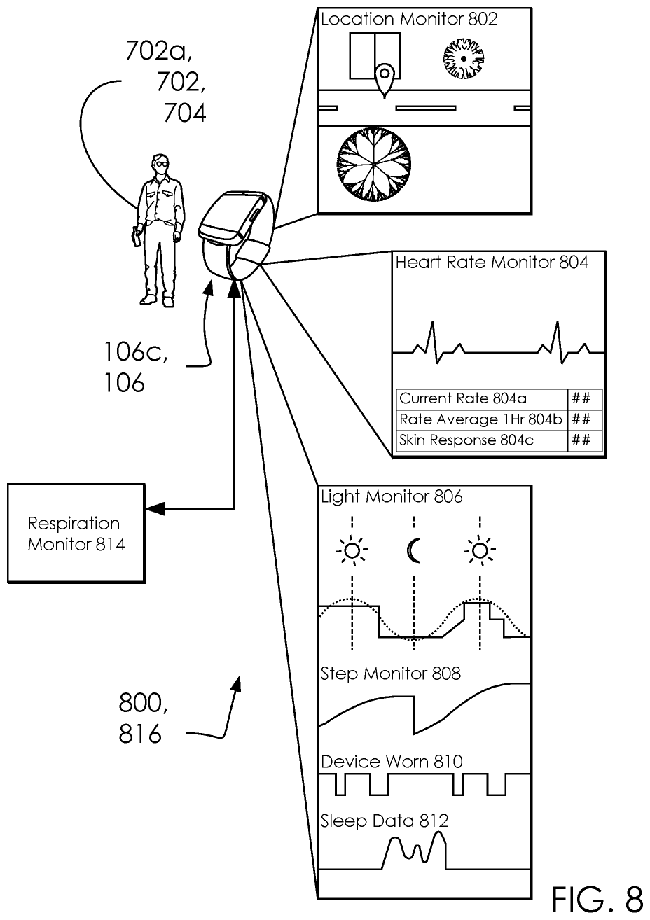

FIG. 8 illustrates a flow chart view of a monitoring data illustration 800.

In one embodiment, said monitoring data illustration 800 can comprise a wearable health data 816 comprising a location monitor data 802, a heart rate monitor data 804, a light monitor data 806, a step monitor data 808, a device worn data 810, a sleep data 812, and a respiration monitor 814.

In one embodiment, said heart rate monitor data 804 can comprise a current rate data 804a, an average rate data 804b and a skin response 804c.

Said monitoring system 100 can record said wearable health data 816 using sensors on said wearable mobile computer 106c. whereafter, said wearable health data 816 can be transmitted to said server application 506 for processing.

In one embodiment, said respiration monitor 814 can be attached to said patient user group 704 to track respiration rates and similar data among said wearable health data 816.

In one embodiment, said average rate data 804b can be useful in tracking the change in signals among said monitoring data illustration 800. Said monitoring system 100 can be adapted at tracking changes (delta) in said wearable health data 816 to predict a relapse or pre-relapse behavior.

In one embodiment, said location monitor data 802 can be tracked to determine whether a user is in a good zone or a bad zone. For example, in one embodiment, a bar or red-light district might be considered a bad zone; and, a scheduled AA meeting or accountability partner's home might be considered a safe zone or good zone.

In one embodiment, said skin response 804c can comprise a galvanic skin response. In one embodiment, said heart rate monitor data 804 can be tracked with a light sensor, as is known in the art, for tracking heart rate and skin conditions.

In one embodiment, said light monitor data 806 can comprise a light sensor for tracking ambient light around said wearable mobile computer 106c, as is known in the art.

In one embodiment, said step monitor data 808 can comprise a step counter as is known in the art.

In one embodiment, said device worn data 810 can comprise a binary signal whether said wearable mobile computer 106c is being worn.

Said sleep data 812 can track qualify a quality of sleep for said patient user group 704, as is known in the art.

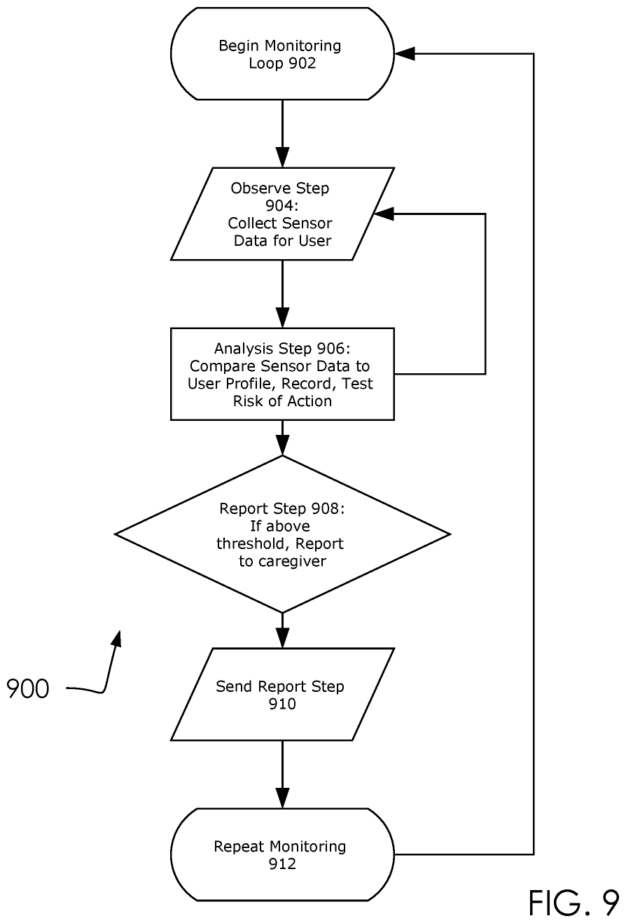

FIG. 9 illustrates a flow chart view of monitoring procedure 900.

In one embodiment, said monitoring system 100 can comprise a monitoring procedure 900, a begin monitor loop step 902, an observe step 904, an analysis step 906, a report step 908, a send report step 910, a report monitoring step 912, a sensor data 914 and a user profile 916.

In one embodiment, said monitoring system 100 can be configured to monitor the status of said one or more users 702. For example, in one embodiment, said monitoring procedure 900 can illustrate monitoring said wearable health data 816; wherein: said observe step 904 can comprise collecting said wearable health data 816; said analysis step 906 can comprise looking through said wearable health data 816 for indicators of relapse or general risk; said report step 908 can comprise reporting and/or logging on a dangerous condition; said send report step 910 can comprise sending said report to other users of said monitoring system 100; and said report monitoring step 912 can comprise repeating the steps of said monitoring procedure 900.

Many methods of observing, analyzing and monitoring users of said monitoring system 100 are disclosed herein.

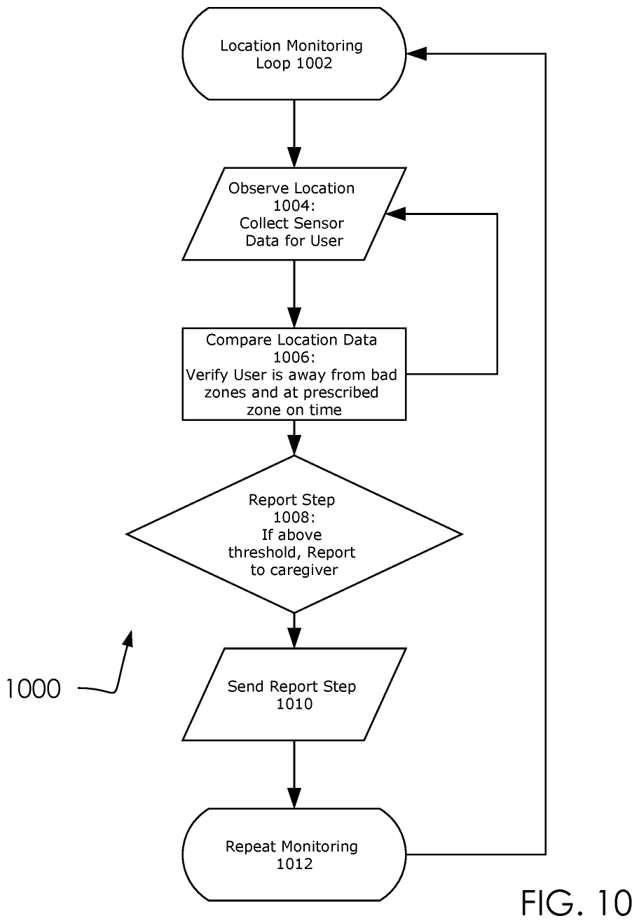

FIG. 10 illustrates a flow chart view of location monitoring procedure 1000.

In one embodiment, said data 206 can comprise a user location data 1014, a blacklist locations 1016 and a whitelist locations 1018.

In one embodiment, said monitoring system 100 can comprise a location monitoring procedure 1000, a location monitoring loop start step 1002, an observe location step 1004, a compare location data step 1006, a report step 1008, a send report step 1010 and a repeat monitoring step 1012.

Said location monitoring procedure 1000 can comprise a summary of steps for monitoring user locations and using that data to serve users of said monitoring system 100.

Said location monitoring loop start step 1002 can comprise beginning said location monitoring procedure 1000; said observe location step 1004 can comprise collecting said user location data 1014; said compare location data step 1006 can comprise comparing said user location data 1014 to said blacklist locations 1016 and said whitelist locations 1018; said report step 908 1008 comprises determining if the users' location is within said user location data 1014 or said blacklist locations 1016; said send report step 910 1010 can comprise reporting on the status of a user as discussed herein; and said repeat monitoring step 1012 can comprise repeating said location monitoring procedure 1000.

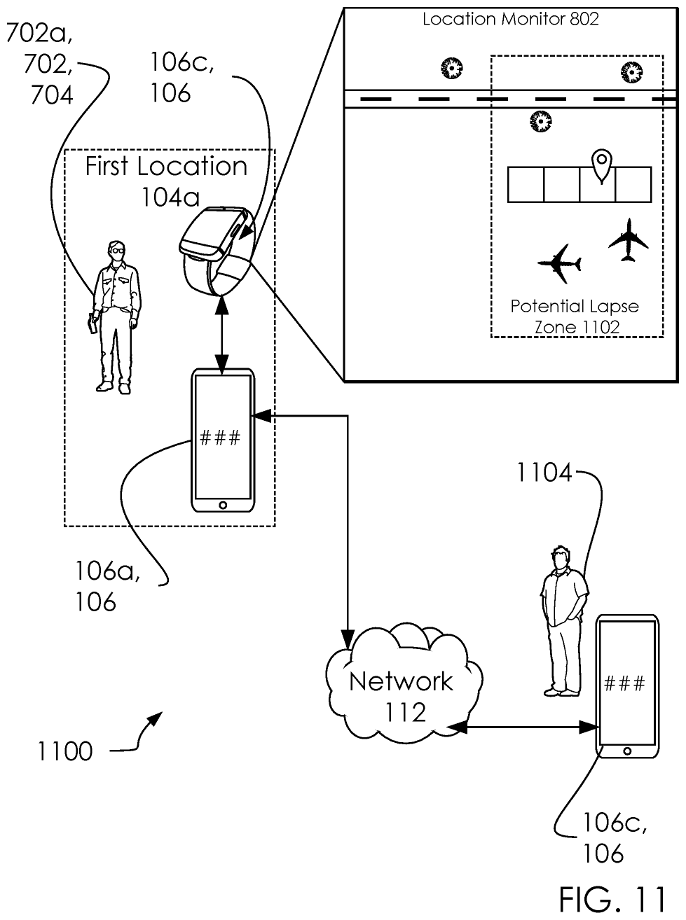

FIG. 11 illustrates a view of a location monitoring flowchart 1100.

In one embodiment, said location monitoring procedure 1000 can comprise a grey-list potential lapse zone 1102.

In one embodiment, said one or more users 702 can comprise a friends-group 1104.

In one embodiment, said monitoring system 100 can monitor said patient user group 704 with said location monitoring procedure 1000. Wherein, said observe location step 1004 can comprise monitoring to see if that user has entered a bad zone or danger zone, such as an airport (a common place of relapse). Wherein, said monitoring system 100 can send a message to said therapist user group 706, said accountability user group 708, and said caregiver user group 710, or other persons providing help to said patient user group 704. Further, a message can play on said one or more locations 104 and/or said one or more mobile computers 106 for said patient user group 704 to encourage them.

For example, in one embodiment, a first patient 704a enters said grey-list potential lapse zone 1102 (in this example, the airport), said monitoring system 100 sends a pre-recorded message of a loved one in said friends-group 1104 encouraging said first patient 704a to said first mobile computer 106a saying that said first patient 704a is loved and how proud his family is of him. In one embodiment, a goal of such a message may be to snap the mind of a member of said patient user group 704 back into strength in order to power through temptation.

Likewise, said grey-list potential lapse zone 1102 may be marked by merely altering settings on said one or more mobile computers 106. For example, if said first mobile computer 106a is turned into airplane mode (for flight time), said monitoring system 100 can display a message, as discussed herein, for the encouragement of said first user 702a.

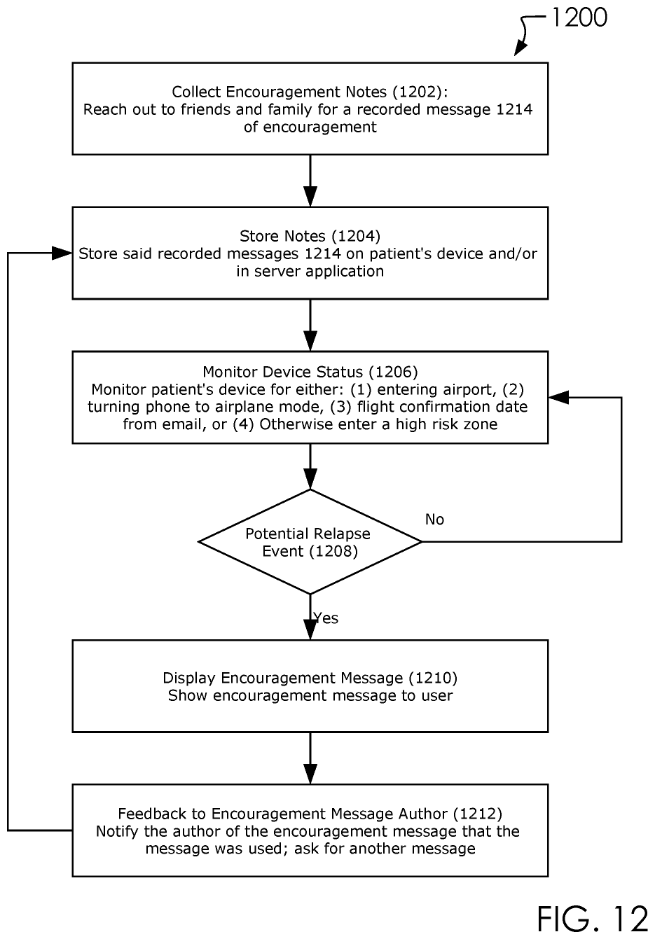

FIG. 12 illustrates a view of an encouragement method 1200.

In one embodiment, said encouragement method 1200 can comprise a notes collection step 1202, a notes storage step 1204, a monitoring step 1206, a risk assessment step 1208, an encouragement display step 1210, a feedback step 1212 and a recorded message 1214.

In one embodiment, said monitoring system 100 can comprise said encouragement method 1200.

In one embodiment, said monitoring system 100 can further comprise the capability of encouraging said patient user group 704 at a time of need. For example, in one embodiment, said encouragement method 1200 can comprise steps enabled by said monitoring system 100 for such a need; wherein: said notes collection step 1202 can comprise reaching out to said friends-group 1104 (such as family, friends and care givers to said patient user group 704) to request said recorded message 1214; said notes storage step 1204 can comprise storing said recorded message 1214; said monitoring step 1206 can comprise storing said recorded message 1214 on said first mobile computer 106a; said monitoring step 1206 can comprise monitoring said first mobile computer 106a for entry into a travel location such as an airport, putting said first mobile computer 106a into an active but disconnected state such as airplane mode, by reviewing flight bookings in email or calendar, or by passing near a potential relapse zone (such as those in said blacklist locations 1016); said risk assessment step 1208 can comprise assessing whether the potential relapse event has occurred; said encouragement display step 1210 can comprise displaying said recorded message 1214 to said patient user group 704; and said feedback step 1212 can comprise notifying the author of said recorded message 1214 of this event and/or asking for additional messages to be used in the future. In one embodiment, said notes collection step 1202 can be triggered when said device application 502 and/or said server 108 application 506 become aware that said patient user group 704 has a flight booked.

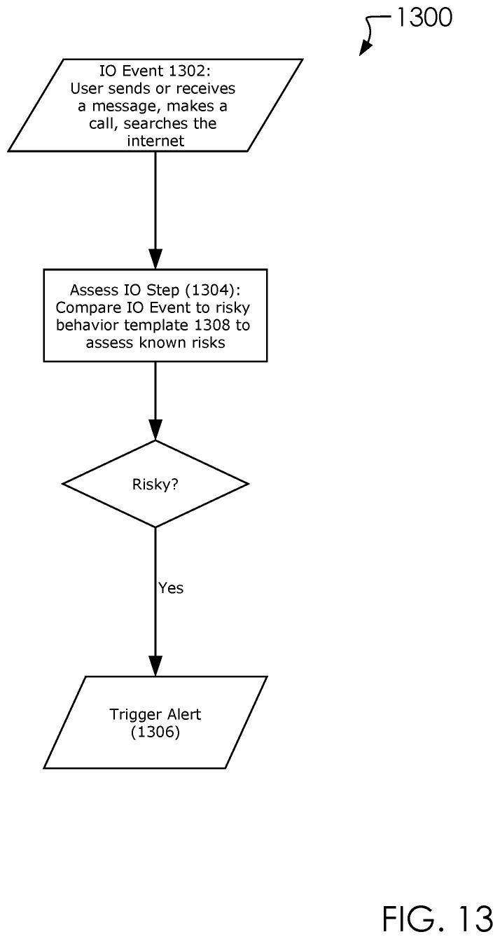

FIG. 13 illustrates a view of an IO Event monitoring method 1300.

In one embodiment, said IO Event monitoring method 1300 can comprise an IO event 1302, said assess said IO event 1302 step assess said IO event step 1304, a trigger alert 1306 and a risky behavior template 1308.

In one embodiment, said monitoring system 100 can comprise said IO Event monitoring method 1300.

In one embodiment, said monitoring system 100 can comprise a method for monitoring in and out events on said one or more mobile computers 106 for said patient user group 704. For example, said IO Event monitoring method 1300 can comprise exemplary steps wherein: said IO event 1302 can comprise monitoring emails, texts, messages, internet searches and the like; said assess said IO event step 1304 can comprise comparing said IO event 1302 to said risky behavior template 1308; and said trigger alert 1306 can comprise alerting a portion of the users of said monitoring system 100 according to this disclosure.

Examples of said risky behavior template 1308 can comprise entering an area such as said blacklist locations 1016 or said grey-list potential lapse zone 1102, discussed above.

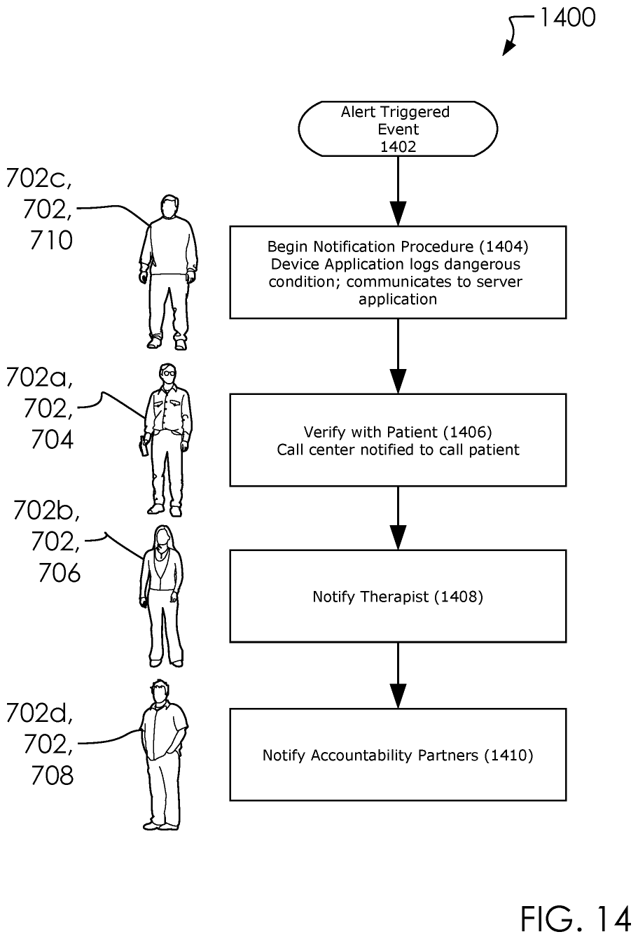

FIG. 14 illustrates a view of a notification method 1400.

In one embodiment, said notification method 1400 can comprise an alert triggered event 1402, a begin notification steps 1404, a verify with patient step 1406, a notify therapist step 1408 and a notify accountability partners step 1410.

In one embodiment, said monitoring system 100 can comprise said notification method 1400.

In one embodiment, said notification method 1400 can comprise a procedure for notifying a portion of said one or more users 702 of said monitoring system 100 when said alert triggered event 1402 occurs, wherein: said begin notification steps 1404 can comprise logging the event in said device application 502 and/or said server 108 application 506; said verify with patient step 1406 can comprise calling said patient user group 704 to check on their status; said notify therapist step 1408 can comprise notifying a portion of said therapist user group 706; and said notify accountability partners step 1410 can comprise notifying a portion of said accountability user group 708.

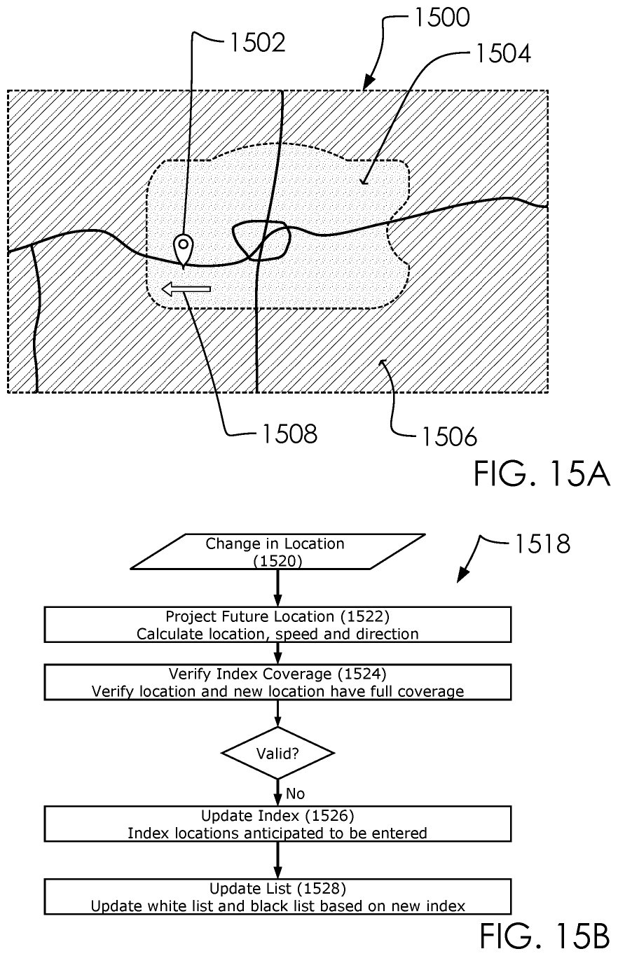

FIGS. 15A and 15B illustrate a view of an index geographical chart 1500 and a dynamic index update procedure 1518.

In one embodiment, said index geographical chart 1500 can comprise a user location 1502, a valid indexed area 1504, an invalid indexed area 1506 and a user travel parameters 1508.

In one embodiment, said monitoring system 100 can comprise said dynamic index update procedure 1518. Said dynamic index update procedure 1518 can comprise a receive a change in user location event 1520, a project future user location step 1522, a verify index coverage step 1524, an update index step 1526 and an update lists step 1528.

In one embodiment, said monitoring system 100 can comprise said index geographical chart 1500 and said dynamic index update procedure 1518.

In one embodiment, said dynamic index update procedure 1518 can comprise verifying that said index geographical chart 1500 is valid at all times. Said monitoring system 100 can comprise said blacklist locations 1016 and said whitelist locations 1018 based on an index of data systems such as locations of liquor stores, casinos, red light districts, or other known locations of relapse. In one embodiment, said monitoring system 100 can crawl the web to index such locations, phone numbers, characteristics and the like. However, where said monitoring system 100 has an inadequate index of a location, such as at said invalid indexed area 1506 (as illustrated), said monitoring system 100 may need to further index said invalid indexed area 1506 prior to the arrival of said patient user group 704.

For example, said dynamic index update procedure 1518 can be configured to track said user travel parameters 1508 and said user location 1502 to anticipate temptations of said patient user group 704: said receive a change in user location event 1520 can comprise a change in the location of said patient user group 704 with said one or more mobile computers 106; said project future user location step 1522 can comprise calculating said user travel parameters 1508; said verify index coverage step 1524 can comprise verifying said user travel parameters 1508 won't take said patient user group 704 outside of said valid indexed area 1504 within a specified time; and said update index step 1526 and said update lists step 1528 can comprise updating said blacklist locations 1016 and said whitelist locations 1018 for a portion of said invalid indexed area 1506.

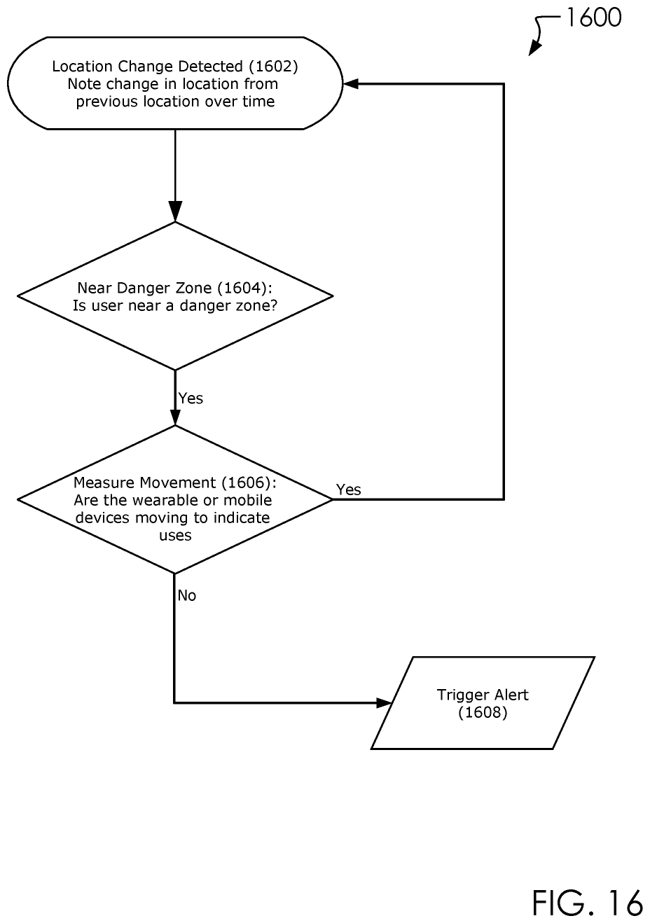

FIG. 16 illustrates a view of a motion detection method 1600.

In one embodiment, said motion detection method 1600 can comprise a location detection event 1602, a near danger zone assessment 1604, a measure user movement step 1606 and said trigger alert 1306 step 1608.

In one embodiment, said monitoring system 100 can comprise said motion detection method 1600.

In one embodiment, said motion detection method 1600 can ensure that said patient user group 704 doesn't remove said first mobile computer 106a and/or said wearable mobile computer 106c while near a location in said blacklist locations 1016 and/or said grey-list potential lapse zone 1102; accordingly, said monitoring system 100 is configured to ensure active use of said monitoring system 100 when near danger. Said patient user group 704 is thereby discouraged from removing said wearable mobile computer 106c and running into a liquor store for a quick purchase, or similar quick scores.

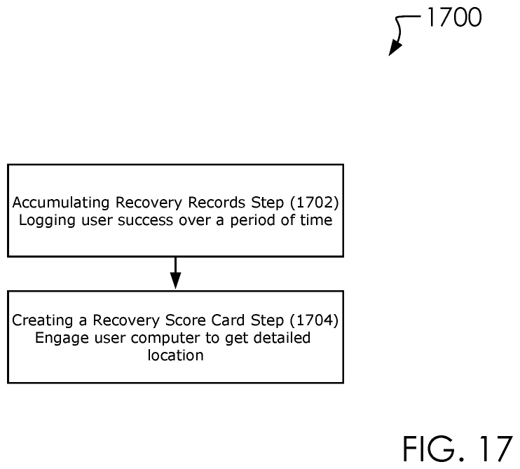

FIG. 17 illustrates a view of a method of creating a recovery score card 1700.

In one embodiment, said method of creating a recovery score card 1700 can comprise an accumulating records step 1702 and a creating a recovery score card step 1704.

In one embodiment, said monitoring system 100 can comprise said method of creating a recovery score card 1700.

In one embodiment, said monitoring system 100 can use said method of creating a recovery score card 1700 to create a recovery scorecard 1802.

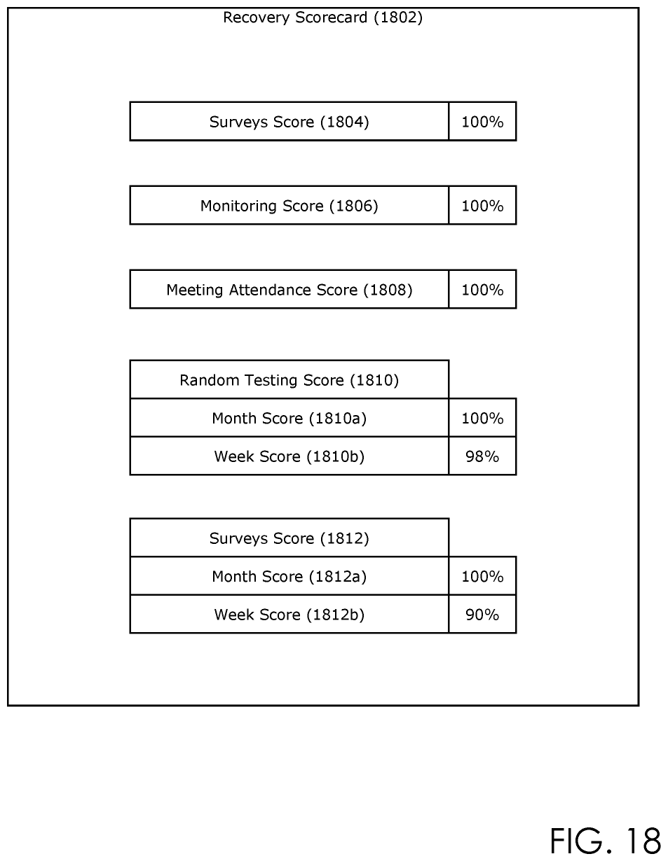

FIG. 18 illustrates a view of said recovery scorecard 1802.

In one embodiment, said recovery scorecard 1802 can comprise a surveys score 1804, a monitoring score 1806, a meeting attendance score 1808, said meeting attendance score 1808, one or more random testing scores 1810, a second random testing score 1810b and one or more surveys scores 1812.

In one embodiment, said one or more random testing scores 1810 can comprise a first random testing score 1810a and said second random testing score 1810b.

In one embodiment, said one or more surveys scores 1812 can comprise a first surveys score 1812a and a second surveys score 1812b.

In one embodiment, said monitoring system 100 can comprise said recovery scorecard 1802.

In one embodiment, said recovery scorecard 1802 can be displayed to said one or more users 702 for encouragement and accountability purpose.

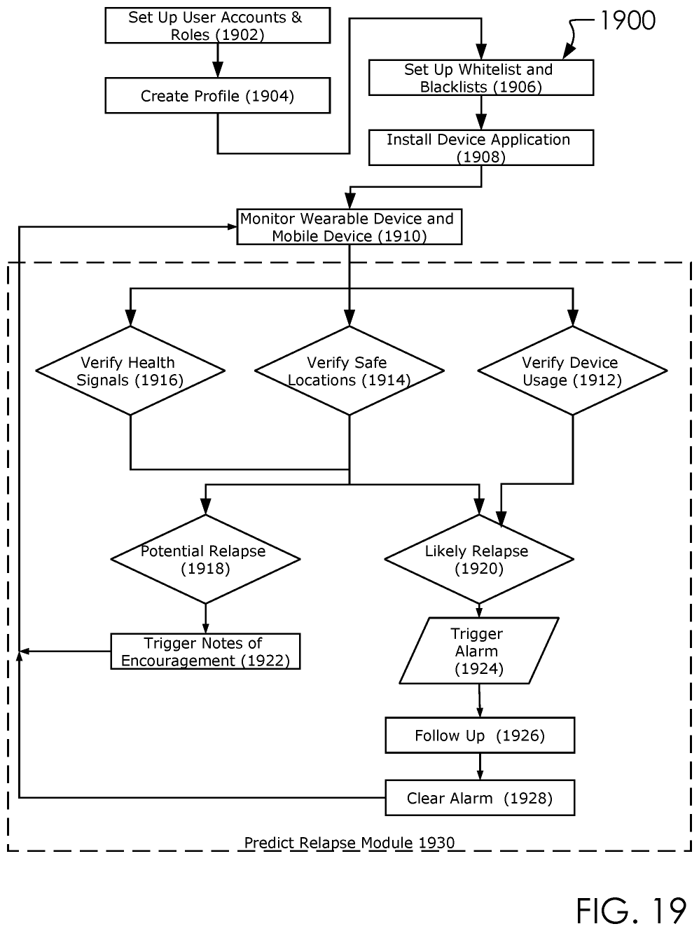

FIG. 19 illustrates a view of an overview chart 1900.

In one embodiment, said overview chart 1900 can comprise a set up users and roles step 1902, a create profile step 1904, a set up whitelist and blacklist step 1906, an install device application step 1908, a monitor wearables and mobile devices step 1910, a verify device usage method 1912, a verify sage locations method 1914, a verify health signals method 1916, a measure potential relapse method 1918, a measure likely relapse method 1920, a triggering notes of encouragement step 1922, a triggering alarm step 1924, a follow up step 1926 and a clear alarm step 1928.

In one embodiment, said monitoring system 100 can comprise said overview chart 1900.

Said overview chart 1900 can comprise an overview of said monitoring system 100 in a simplified flow chart.

Steps 1916-1928 can be referred to as a predict relapse module 1930.

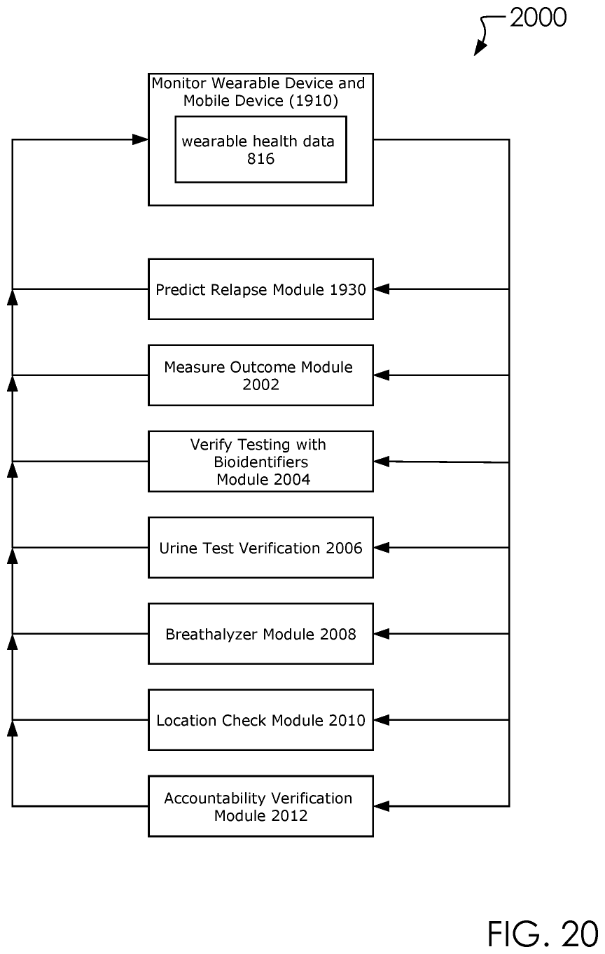

FIG. 20 illustrates an integrity ally module 2000 of said monitoring system 100.

Modules within said integrity ally module 2000 can comprise said predict relapse module 1930, a measure outcome module 2002, a verify testing through bio-identifiers module 2004, a urine test verification module 2006, a breathalyzer module 2008, a location check module 2010, and an accountability verification module 2012.

Said monitoring system 100 can be configured to monitor said wearable health data 816 and implement said integrity ally module 2000.

Said integrity ally module 2000 can be configured for monitoring said wearable health data 816 in step said monitor wearables and mobile devices step 1910.

Said measure outcome module 2002 can compare predictions with results over time of said wearable health data 816 to further refine predictions made by said integrity ally module 2000.

Said verify testing through bio-identifiers module 2004 can comprise requesting thumbprints, facial recognition, privacy data, or similar when accepting feedback from said patient user group 704.

Said urine test verification module 2006 can comprise directing said patient user group 704 to verified clinics for urine testing. Wherein, test results are collected, analyzed and uploaded by verified/trusted personnel.

Said breathalyzer module 2008 can comprise a Bluetooth® configured device for collecting breath samples of users to verify nonintoxicating. Similar paired devices are known in the art and would be acceptable as an alternative to Bluetooth.

Said location check module 2010 can comprise features discussed in said location monitoring procedure 1000 and said location monitoring flowchart 1100.

Said accountability verification module 2012 can comprise verification of attendance at group meetings with said accountability user group 708, verification that said wearable mobile computer 106c is being worn and logging said wearable health data 816 in said monitoring system 100.

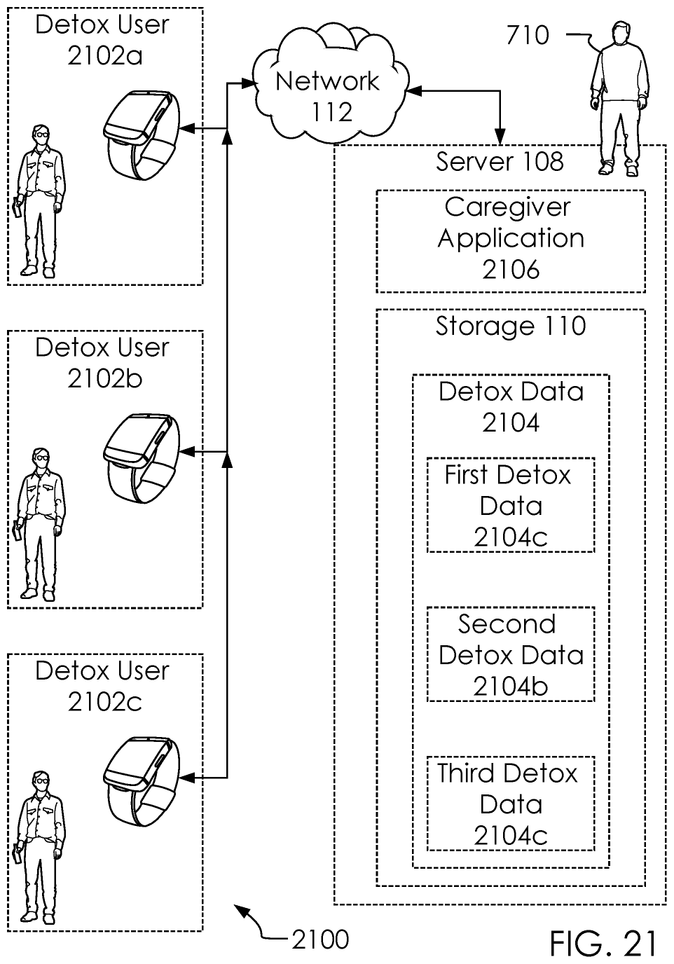

FIG. 21 illustrates a detox ally module 2100 as a flow chart.

Said monitoring system 100 can further comprise said detox ally module 2100 and a caregiver application 2106.

Said detox ally module 2100 can comprise a plurality of detox users 2102 among said patient user group 704. Said plurality of detox users 2102 can comprise first detox users 2102a, second detox users 2102b, and third detox users 2102c.

Said detox ally module 2100 can comprise collecting a detox data 2104 from among said patient user group 704. Said detox data 2104 can comprise a first detox data 2104a, a second detox data 2104b, and a third detox data 2104c, each associated with one among said plurality of detox users 2102.

In one embodiment, said patient user group 704 may go through a visit to a detox clinic prior to a rehab clinic. Wherein, said caregiver user group 710 can comprise a nurse or licensed medical professional tasked with monitoring a plurality of users, such as said plurality of detox users 2102. In one embodiment, said monitoring system 100 can collect said wearable health data 816 for said plurality of detox users 2102 and report said detox data 2104 to said caregiver application 2106 so that said caregiver user group 710 can keep a keen eye on each among said network diagram 102.

One feature of said wearable health data 816 which can be monitored by said detox ally module 2100 can comprise stabilization of health metrics such as said heart rate monitor data 804, said current rate data 804a, said average rate data 804b, said skin response 804c, and said light monitor data 806.

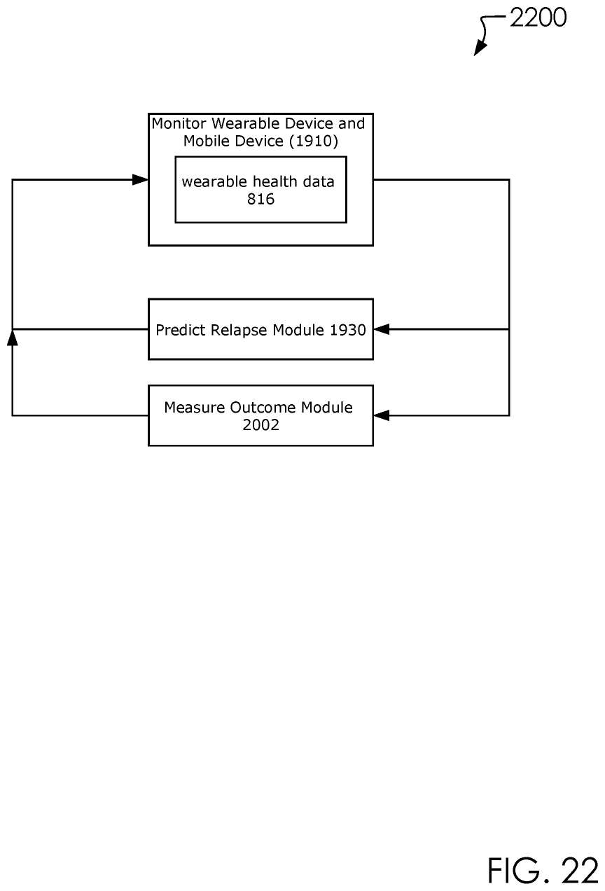

FIG. 22 illustrates a protective ally module 2200 of said monitoring system 100.

In one embodiment, said monitoring system 100 can comprise said protective ally module 2200 for monitoring health signals for said patient user group 704 in cases where said patient user group 704 needs to be kept stable. Examples of these circumstances include drug overdoses, those who want to quit a drug, or similar.

In such cases said protective ally module 2200 can comprise monitoring said wearable mobile computer 106c, which can comprise a skin patch comprising a PCB and communication hardware, rather than a smartwatch. Said patch can comprise a multi-day battery and can be either reapplied or replaced periodically. In one embodiment, said protective ally module 2200 can monitor a blood oxygen data 2202, said heart rate monitor data 804, and said respiration monitor 814.

These parts are illustrated in the figures and discussed below:
said network diagram 102,
said one or more locations 104,
said one or more mobile computers 106,
said server 108,
said data storage 110,
said network 112,
said printer 114,
said first mobile computer 106a,
said second mobile computer 106b,
said wearable mobile computer 106c,
said first location 104a,
said second location 104b,
said third location 104c,
said monitoring center 104d,
said data storage 110a,
said monitoring system 100,
said one or more wearable computers 116,
said first wearable computer 116a,
said second wearable computer 116b,
said mobile phone 200a,
said personal computer 200b,
said tablet 200c,
said smartphone 222,
said screen 202,
said data 206,
said home button 220,
said one or more input devices 204,
said keyboard 204a,
said trackball 204b,
said one or more cameras 204c,
said track pad 204d,
said smart watch 200d,
said smart phone 200e,
said address space 302,
said address space 302a,
said address space 302e,
said processor 304,
said memory 306,
said communication hardware 308,
said location hardware 310,
said processor 304a,
said memory 306a,
said communication hardware 308a,
said processor 304e,
said communication hardware 306e,
said memory 306e,
said communication hardware 308e,
said address space 302d,
said device application 502,
said server application 506,
said memory 306d,
said data records 504a,
said one or more users 702,
said first user 702a,
said second user 702b,
said third user 702c,
said fourth user 702d,
said patient user group 704,
said therapist user group 706,
said accountability user group 708,
said caregiver user group 710,
said monitoring data illustration 800,
said wearable health data 816,
said location monitor data 802,
said heart rate monitor data 804,
said light monitor data 806,
said step monitor data 808,
said device worn data 810,
said sleep data 812,
said respiration monitor 814,
said current rate data 804a,
said average rate data 804b,
said skin response 804c,
said monitoring procedure 900,
said begin monitor loop step 902,
said observe step 904,
said analysis step 906,
said report step 908,
said send report step 910,
said report monitoring step 912,
said sensor data 914,
said user profile 916,
said user location data 1014,
said blacklist locations 1016,
said whitelist locations 1018,
said location monitoring procedure 1000,
said location monitoring loop start step 1002,
said observe location step 1004,
said compare location data step 1006,
said report step 1008,
said send report step 1010,
said repeat monitoring step 1012,
said location monitoring flowchart 1100,
said grey-list potential lapse zone 1102,
said friends-group 1104,
said first patient 704a,
said encouragement method 1200,
said notes collection step 1202,
said notes storage step 1204,
said monitoring step 1206,
said risk assessment step 1208,
said encouragement display step 1210,
said feedback step 1212,
said recorded message 1214,
said IO Event monitoring method 1300,
said IO event 1302,
said assess said IO event step 1304,
said trigger alert 1306,
said risky behavior template 1308,
said notification method 1400,
said alert triggered event 1402,
said begin notification steps 1404,
said verify with patient step 1406,
said notify therapist step 1408,
said notify accountability partners step 1410,
said index geographical chart 1500,
said user location 1502,
said valid indexed area 1504,
said invalid indexed area 1506,
said user travel parameters 1508,
said dynamic index update procedure 1518,
said receive a change in user location event 1520,
said project future user location step 1522,
said verify index coverage step 1524,
said update index step 1526,
said update lists step 1528,
said motion detection method 1600,
said location detection event 1602,
said near danger zone assessment 1604,
said measure user movement step 1606,
said method of creating a recovery score card 1700,
said accumulating records step 1702,
said creating a recovery score card step 1704,
said recovery scorecard 1802,
said surveys score 1804,
said monitoring score 1806,
said meeting attendance score 1808,
said one or more random testing scores 1810,
said second random testing score 1810b,
said one or more surveys scores 1812,
said first random testing score 1810a,
said first surveys score 1812a,
said second surveys score 1812b,
said overview chart 1900,
said set up users and roles step 1902,
said create profile step 1904,
said set up whitelist and blacklist step 1906,
said install device application step 1908,
said monitor wearables and mobile devices step 1910,
said verify device usage method 1912,
said verify sage locations method 1914,
said verify health signals method 1916,
said measure potential relapse method 1918,
said measure likely relapse method 1920,
said triggering notes of encouragement step 1922,
said triggering alarm step 1924,
said follow up step 1926,
said clear alarm step 1928,
said predict relapse module 1930,
said integrity ally module 2000,
said measure outcome module 2002,
said verify testing through bio-identifiers module 2004,
said urine test verification module 2006,
said breathalyzer module 2008,
said location check module 2010,
said accountability verification module 2012,
said detox ally module 2100,
said caregiver application 2106,
said plurality of detox users 2102,
said first detox users 2102a,
said second detox users 2102b,
said third detox users 2102c,
said detox data 2104,
said first detox data 2104a,
said second detox data 2104b,
said third detox data 2104c,
said protective ally module 2200, and
said blood oxygen data 2202.

The following sentences represent at least one preferred embodiment based on the original claims.

Said monitoring system 100 for assessing a condition of and facilitating treatment of said patient user group 704 in an addiction treatment program. Said monitoring system 100 comprises at least said wearable mobile computer 106c, said server 108, said device application 502 and said server application 506. Said device application 502 can be on said wearable mobile computer 106c. Said server application 506 can be on said server 108. Said server application 506 comprises at least said caregiver application 2106 for reporting a status of said patient user group 704 to said caregiver user group 710 of said monitoring system 100. Said caregiver user group 710 can comprise therapists and accountability partners. Said monitoring system 100 collects said wearable health data 816 for each among said patient user group 704 which can be wearing said wearable mobile computer 106c. Said monitoring system 100 can record said wearable health data 816 using sensors on said wearable mobile computer 106c. Said monitoring system 100 can be adapted at tracking changes in said wearable health data 816 to predict a relapse or pre-relapse behavior. Said monitoring system 100 can be configured to report a portion of said wearable health data 816 to said caregiver application 2106 and thereafter to a portion of said caregiver user group 710. Said wearable health data 816 comprises a portion of said location monitor data 802, said heart rate monitor data 804, said light monitor data 806, said step monitor data 808, said device worn data 810, said sleep data 812, and said respiration monitor 814. Said respiration monitor 814 can be attached to said patient user group 704 to track respiration rates and similar data among said wearable health data 816.

Said monitoring system 100 for assessing a condition of and facilitating treatment of said patient user group 704 in an addiction treatment program. Said monitoring system 100 comprises at least said wearable mobile computer 106c, said server 108, said device application 502 and said server application 506. Said device application 502 can be on said wearable mobile computer 106c. Said server application 506 can be on said server 108. Said server application 506 comprises at least said caregiver application 2106 for reporting a status of said patient user group 704 to said caregiver user group 710 of said monitoring system 100. Said caregiver user group 710 can comprise therapists and accountability partners. Said monitoring system 100 collects said wearable health data 816 for each among said patient user group 704 which can be wearing said wearable mobile computer 106c. Said monitoring system 100 can record said wearable health data 816 using sensors on said wearable mobile computer 106c. Said monitoring system 100 can be adapted at tracking changes in said wearable health data 816 to predict a relapse or pre-relapse behavior. Said monitoring system 100 can be configured to report a portion of said wearable health data 816 to said caregiver application 2106 and thereafter to a portion of said caregiver user group 710. Said wearable health data 816 comprises a portion of said location monitor data 802, said heart rate monitor data 804, said light monitor data 806, said step monitor data 808, said device worn data 810, said sleep data 812, and said respiration monitor 814. Said respiration monitor 814 can be attached to said patient user group 704 to track respiration rates and similar data among said wearable health data 816.

Said monitoring system 100 for assessing a condition of and facilitating treatment of said patient user group 704 in an addiction treatment program. Said monitoring system 100 comprises at least said wearable mobile computer 106c, said server 108, said device application 502 and said server application 506. Said device application 502 can be on said wearable mobile computer 106c. Said server application 506 can be on said server 108. Said server application 506 comprises at least said caregiver application 2106 for reporting a status of said patient user group 704 to said caregiver user group 710 of said monitoring system 100. Said caregiver user group 710 can comprise therapists and accountability partners. Said monitoring system 100 collects said wearable health data 816 for each among said patient user group 704 which can be wearing said wearable mobile computer 106c. Said monitoring system 100 can record said wearable health data 816 using sensors on said wearable mobile computer 106c. Said monitoring system 100 can be adapted at tracking changes in said wearable health data 816 to predict a relapse or pre-relapse behavior. Said monitoring system 100 can be configured to report a portion of said wearable health data 816 to said caregiver application 2106 and thereafter to a portion of said caregiver user group 710.

A portion of said patient user group 704 comprise said plurality of detox users 2102.

Said wearable health data 816 can be transmitted to said server application 506 for processing.

Said wearable health data 816 comprises a portion of said location monitor data 802, said heart rate monitor data 804, said light monitor data 806, said step monitor data 808, said device worn data 810, said sleep data 812, and said respiration monitor 814. Said respiration monitor 814 can be attached to said patient user group 704 to track respiration rates and similar data among said wearable health data 816.

Said heart rate monitor data 804 comprises said current rate data 804a, said average rate data 804b and said skin response 804c.

Said location monitor data 802 can be tracked to determine whether a user can be in a good zone or a bad zone. A bar or red-light district might be considered a bad zone., a scheduled AA meeting or accountability partner's home might be considered a safe zone or good zone.

Said skin response 804c comprises a galvanic skin response.

Said heart rate monitor data 804 can be tracked with a light sensor for tracking heart rate and skin conditions.

Said light monitor data 806 comprises a light sensor for tracking ambient light around said wearable mobile computer 106c.

Said device worn data 810 comprises a binary signal whether said wearable mobile computer 106c can be being worn.

Said monitoring system 100 can be configured for monitoring the status of said wearable health data 816 of said one or more users 702 among said patient user group 704 by collecting said wearable health data 816, looking through said wearable health data 816 for indicators of relapse or general risk, logging on a dangerous condition. Reporting on a dangerous condition, and sending said report to other users of said monitoring system 100.

Said wearable health data 816 comprises said user location data 1014, said blacklist locations 1016 and said whitelist locations 1018. Said monitoring system 100 can be configured for collecting said user location data 1014. Comparing said user location data 1014 to said blacklist locations 1016 and said whitelist locations 1018. Determining if the users' location can be within said user location data 1014 or said blacklist locations 1016. Reporting on the status of a user.

Said monitoring system 100 can be configured for encouraging said patient user group 704 at a time of need by reaching out to said friends-group 1104 to request said recorded message 1214, storing said recorded message 1214 on said first mobile computer 106a, monitoring said first mobile computer 106a for one among entry into a travel location, putting said first mobile computer 106a into an active but disconnected state such as airplane mode, for flight bookings in email or calendar, or by passing near a potential relapse zone in said blacklist locations 1016, and displaying said recorded message 1214 to said patient user group 704.

Said monitoring system 100 can be configured for encouraging said patient user group 704 at a time of need by reaching out to said friends-group 1104 to request said recorded message 1214, storing said recorded message 1214 on said first mobile computer 106a, assessing whether the potential relapse event has occurred, and displaying said recorded message 1214 to said patient user group 704.

Said monitoring system 100 for assessing a condition of and facilitating treatment of said patient user group 704 in an addiction treatment program. Said monitoring system 100 comprises at least said wearable mobile computer 106c, said server 108, said device application 502 and said server application 506. Said device application 502 can be on said wearable mobile computer 106c. Said server application 506 can be on said server 108. Said server application 506 comprises at least said caregiver application 2106 for reporting a status of said patient user group 704 to said caregiver user group 710 of said monitoring system 100. Said caregiver user group 710 can comprise therapists and accountability partners. Said monitoring system 100 collects said wearable health data 816 for each among said patient user group 704 which can be wearing said wearable mobile computer 106c. Said monitoring system 100 can record said wearable health data 816 using sensors on said wearable mobile computer 106c. Said monitoring system 100 can be adapted at tracking changes in said wearable health data 816 to predict a relapse or pre-relapse behavior. Said monitoring system 100 can be configured to report a portion of said wearable health data 816 to said caregiver application 2106 and thereafter to a portion of said caregiver user group 710. Said wearable health data 816 comprises said user location data 1014, said blacklist locations 1016 and said whitelist locations 1018. Said monitoring system 100 can be configured for collecting said user location data 1014. Comparing said user location data 1014 to said blacklist locations 1016 and said whitelist locations 1018. Determining if the users' location can be within said user location data 1014 or said blacklist locations 1016. Reporting on the status of a user.

1. A monitoring system for assessing a condition of and facilitating treatment of a patient user group in an addiction treatment program, wherein:
said monitoring system comprises at least a wearable mobile computer, a server, a device application and a server application;
said device application is on said wearable mobile computer;
said server application is on said server;
said server application comprises at least a caregiver application for reporting a status of said patient user group to a caregiver user group of said monitoring system;
said caregiver user group can comprise therapists and accountability partners;
said monitoring system collects a wearable health data for each among said patient user group which are wearing said wearable mobile computer;
said monitoring system can record said wearable health data using sensors on said wearable mobile computer;
said monitoring system is adapted at tracking changes in said wearable health data to predict a relapse or pre-relapse behavior; and
said monitoring system is configured to report a portion of said wearable health data to said caregiver application and thereafter to a portion of said caregiver user group;
said wearable health data comprises a portion of a location monitor data, a heart rate monitor data, a light monitor data, a step monitor data, a device worn data, a sleep data, and a respiration monitor; and
said respiration monitor is attached to said patient user group to track respiration rates and similar data among said wearable health data.

2. A monitoring system for assessing a condition of and facilitating treatment of a patient user group in an addiction treatment program, wherein:
said monitoring system comprises at least a wearable mobile computer, a server, a device application and a server application;
said device application is on said wearable mobile computer;
said server application is on said server;
said server application comprises at least a caregiver application for reporting a status of said patient user group to a caregiver user group of said monitoring system;
said caregiver user group can comprise therapists and accountability partners;
said monitoring system collects a wearable health data for each among said patient user group which are wearing said wearable mobile computer;
said monitoring system can record said wearable health data using sensors on said wearable mobile computer;
said monitoring system is adapted at tracking changes in said wearable health data to predict a relapse or pre-relapse behavior; and
said monitoring system is configured to report a portion of said wearable health data to said caregiver application and thereafter to a portion of said caregiver user group.

3. The monitoring system of claim 2, wherein:
a portion of said patient user group comprise A plurality of detox users.

4. The monitoring system of claim 2, wherein:
said wearable health data is transmitted to said server application for processing.

5. The monitoring system of claim 2, wherein:
said wearable health data comprises a portion of a location monitor data, a heart rate monitor data, a light monitor data, a step monitor data, a device worn data, a sleep data, and a respiration monitor; and
said respiration monitor is attached to said patient user group to track respiration rates and similar data among said wearable health data.

6. The monitoring system of claim 5, wherein:
said heart rate monitor data comprises A current rate data, an average rate data and a skin response.

7. The monitoring system of claim 5, wherein:
said location monitor data is tracked to determine whether a user is in a good zone or a bad zone; and
a bar or red-light district might be considered a bad zone; and, a scheduled AA meeting or accountability partner's home might be considered a safe zone or good zone.

8. The monitoring system of claim 5, wherein:
said skin response comprises a galvanic skin response.

9. The monitoring system of claim 8, wherein:
said heart rate monitor data is tracked with a light sensor for tracking heart rate and skin conditions.

10. The monitoring system of claim 5, wherein:
said light monitor data comprises a light sensor for tracking ambient light around said wearable mobile computer.

11. The monitoring system of claim 5, wherein:
said device worn data comprises a binary signal whether said wearable mobile computer is being worn.

12. The monitoring system of claim 2, wherein:
said monitoring system is configured for monitoring the status of said wearable health data of One or more users among said patient user group by
collecting said wearable health data,
looking through said wearable health data for indicators of relapse or general risk,
logging on a dangerous condition;
reporting on a dangerous condition, and
sending said report to other users of said monitoring system.

13. The monitoring system of claim 2, wherein:
said wearable health data comprises A user location data, a blacklist locations and a whitelist locations;
said monitoring system is configured for
collecting said user location data;
comparing said user location data to said blacklist locations and said whitelist locations;
determining if the users' location is within said user location data or said blacklist locations; and
reporting on the status of a user.

14. The monitoring system of claim 2, wherein:
said monitoring system is configured for encouraging said patient user group at a time of need by
reaching out to a friends-group to request a recorded message,
storing said recorded message on a first mobile computer,
monitoring said first mobile computer for one among entry into a travel location, putting said first mobile computer into an active but disconnected state such as airplane mode, for flight bookings in email or calendar, or by passing near a potential relapse zone in said blacklist locations, and
displaying said recorded message to said patient user group.

15. The monitoring system of claim 2, wherein:
said monitoring system is configured for encouraging said patient user group at a time of need by
reaching out to said friends-group to request said recorded message,
storing said recorded message on said first mobile computer,
assessing whether the potential relapse event has occurred, and
displaying said recorded message to said patient user group.

16. A monitoring system for assessing a condition of and facilitating treatment of a patient user group in an addiction treatment program, wherein:
said monitoring system comprises at least a wearable mobile computer, a server, a device application and a server application;
said device application is on said wearable mobile computer;
said server application is on said server;
said server application comprises at least a caregiver application for reporting a status of said patient user group to a caregiver user group of said monitoring system;
said caregiver user group can comprise therapists and accountability partners;
said monitoring system collects a wearable health data for each among said patient user group which are wearing said wearable mobile computer;
said monitoring system can record said wearable health data using sensors on said wearable mobile computer;
said monitoring system is adapted at tracking changes in said wearable health data to predict a relapse or pre-relapse behavior; and
said monitoring system is configured to report a portion of said wearable health data to said caregiver application and thereafter to a portion of said caregiver user group;
said wearable health data comprises a user location data, a blacklist locations and a whitelist locations;
said monitoring system is configured for
collecting said user location data;
comparing said user location data to said blacklist locations and said whitelist locations;
determining if the users' location is within said user location data or said blacklist locations; and
reporting on the status of a user.

17. The monitoring system of claim 16, wherein:
said monitoring system is configured for encouraging said patient user group at a time of need by
reaching out to a friends-group to request a recorded message,
storing said recorded message on a first mobile computer,
monitoring said first mobile computer for one among entry into a travel location, putting said first mobile computer into an active but disconnected state such as airplane mode, for flight bookings in email or calendar, or by passing near a potential relapse zone in said blacklist locations, and
displaying said recorded message to said patient user group.
